(19) United States
(12) Patent Application Publication
(10) Pub. No.: US 2005/0124957 A1
(43) Pub. Date: Jun. 9, 2005

(54) Means to secure and align absorbent products and maintain fresh feeling for users

(75) Inventor: Tamar Giloh, Salford (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Tamicare Ltd.

(21) Appl. No.: 10/992,418

(22) Filed: Nov. 18, 2004

Related U.S. Application Data
(60) Provisional application No. 60/520,987, filed on Nov. 18, 2003.
(60) Provisional application No. 60/520,989, filed on Nov. 18, 2003.
(60) Provisional application No. 60/520,990, filed on Nov. 18, 2003.

(51) Int. Cl. A61F 13/15; A61F 13/20

(52) U.S. Cl. 604/385.03

(57) ABSTRACT

Stand alone topsheet article that can be engaged over an absorbent article to provide a fresh interface between the user and the absorbent article. An absorbent article with an additional topsheet that can be engaged over the cover of an absorbent article to provide a fresh interface between the user and the absorbent article. The stand alone topsheet or absorbent article is also optionally provided with a cleansing apparatus. The cleansing apparatus is either optionally being packaged with the stand alone topsheet article or absorbent article or integrated into the packaging of the stand alone topsheet article or absorbent article. A device for securing absorbent products is also provided. The device is placed between the absorbent product and an undergarment. The device secures and aligns the absorbent product. As a result, the device reduces the movement of the absorbent product, which may prevent leakage. Finally, a target area marker is provided. The target area marker is used to effectively position an absorbent product. Absorbent products include, but are not limited to, feminine hygiene pads, medical dressings, nursing pads, and diapers. The target area marker may be placed on the absorbent product itself; another article, such as an article of clothing, in which the target area marker is attached; and/or a dedicated marking article, such as a sticker. By effectively placing the absorbent product, the absorption capacity of the product may be maximized.

This application claims the benefit of U.S. Provisional Applications Nos. 60/520,987, filed Nov. 18, 2003; 60/520,989, filed Nov. 18, 2003; and 60/520,990, filed Nov. 18, 2003, the entirety of which is hereby incorporated by reference.

### FIELD OF THE INVENTION

This invention is directed to means for providing a fresh feeling to users of absorbent articles, more particularly to absorbent articles that have an additional topsheet and are optionally packaged with a cleansing apparatus. The invention also relates to absorbent products, and more particularly to a target area marker that indicates where to effectively place the absorbent products. The invention further relates to absorbent products, and more particularly to a device for securing and aligning absorbent products.

### BACKGROUND OF THE INVENTION

It is known in the art that users of absorbent articles can feel uncomfortable after prolonged use of the absorbent article even though the absorbent capabilities of the absorbent article have not been exhausted. This problem exists, for example, for users of absorbent articles for feminine hygiene during the menses. However, this problem also exists for users of other absorbent articles, such as undergarments, absorbent pads, protective undergarments, diapers, nursing pads, and medical dressings. It is also known in the art that users of absorbent articles generally cleanse the area targeted by the absorbent article prior to using an absorbent article and when changing a soiled absorbent article for a fresh one. It is further more known, that many women use very thin absorbent articles like pantiliners daily, to capture feminine discharges, e.g., menses, and maintain fresh feeling. However, these thin pads are sometimes ‘over kill’ for the job. Therefore, it would be beneficial to provide means for establishing a fresh feeling for the users of absorbent articles.

There are also many reasons why an absorbent product may be worn. For example, absorbent products may be worn for feminine hygiene, incontinence, or medical purposes. Frequently, the absorbent product will move out of the desired location. This movement may occur when an individual using the absorbent product moves. For example, an absorbent product attached to the individual's underpants may shift as the individual walks. As a result, leakage may occur.

Typically, adhesive strips are used to hold the absorbent product in place. In addition to the adhesive strips, wings and/or tabs may be added to the absorbent product. For example, the adhesive strips may be used to attach a feminine hygiene pad to the inside crotch section of underpants, while the wings and/or tabs wrap around the edge of the underpants affixing to the outside crotch section of the underpants.

However, the adhesive strips and wings indicate a specific positioning of the feminine hygiene pad according to the underpants and wings design. Unfortunately, this limited variance in position does not always suit a user's body. In addition, the adhesive on the wings tend to stick to the individual's legs. Further, the wings tend to cause the absorbent product to bend into a bridge or inverted “U” shape. This tendency to bend the outer edges of the absorbent product towards the undergarment may cause leakage. Furthermore, being unified with the absorption device, the wings tend to “collect” menses from the absorbent device edges and move them towards the outer side of the undergarment, thus soiling the user's undergarment and garment.

Therefore, it would be beneficial to secure absorbent products. Further, it would be beneficial if the device securing absorbent products can also align the absorbent product in the desired position. By securing and aligning the absorbent product, leakage may be reduced. Furthermore, it is beneficial if the securing device and absorption device are separate articles so that the absorption device having its own sealed edges would force fluids to move longitudinally towards the devices remote areas, and leakage may be reduced. The securing device serves as an additional barrier between the absorption device and the undergarment.

Frequently, the absorbent product is not fully utilized. This lack of utilization may occur when the absorbent product is not placed in the most effective position. For example, absorbent products are frequently attached to undergarments prior to getting dressed. As a result, the absorbent products are not always placed in an accurate position. This may lead to waste, higher costs, and leakage problems. Therefore, it would be beneficial to provide an indication of the most effective position for placing absorbent products.

### SUMMARY OF THE INVENTION

The present invention provides means for users of absorbent articles to establish a fresh feeling prior to using the absorbent article, during prolonged use of the absorbent article, and when interchanging absorbent articles. In one embodiment, the present invention provides a stand alone, attachable, liquid permeable topsheet article to cover absorbent articles, wherein the topsheet contains an adhesive to attach the topsheet to the absorbent article. In another embodiment, the stand alone topsheet can be used to cover absorbent articles such as undergarments, absorbent pads, protective undergarments, diapers, nursing pads, or medical dressings.

In yet another embodiment, the present invention provides a stand alone attachable topsheet article having opposed inwardly and outwardly oriented faces comprising a liquid permeable cover positioned on the inwardly oriented face; a layer of absorption particles positioned on the outwardly oriented face; and an adhesive layer on the outwardly oriented face of the topsheet. In another embodiment, the stand alone topsheet further comprises at least one of a freshening material, an odor-control material, or both. In one embodiment, the stand alone topsheet article can be produced in a white or non-white color.

In another embodiment, the present invention provides an absorbent article comprising: a liquid permeable cover; a liquid impermeable backsheet joined to the cover, said backsheet having opposed inwardly and outwardly oriented faces; an adhesive layer on the outwardly oriented face of the backsheet; an absorption layer positioned between the cover and the backsheet; and a liquid permeable topsheet capable of covering at least a part of the cover.

In another embodiment, the topsheet of the absorbent article is integrated into the absorbent article by fixedly attaching a portion of the topsheet to a portion of the cover. In yet another embodiment, the topsheet of the absorbent article is provided in a condensed state in a new absorbent article, such that the topsheet does not fully cover the cover of the absorbent article. In another embodiment, the topsheet of the absorbent article further comprises a tab for engagement and alignment of the folded topsheet over the cover.

In a further embodiment, the topsheet of the absorbent article contains an adhesive for securing the topsheet to the cover of the absorbent article and optionally contains absorption particles to absorb moisture. In another embodiment, the topsheet of the absorbent article is a fully separable from the absorbent article.

In another embodiment, the additional topsheet optionally contains freshening materials and materials that are capable of absorbing odors.

In another embodiment, the absorbent article is enclosed in a packaging and wherein a cleansing apparatus is optionally provided with the absorbent article. In one embodiment, the optional cleansing apparatus is attached to the packaging for the absorbent article. In another embodiment, the optional cleansing apparatus is integrated into the packaging for the absorbent article.

In yet another embodiment, the topsheet is enclosed in a packaging and wherein a cleansing apparatus is optionally provided with the topsheet. In one embodiment, the optional cleansing apparatus is attached to the packaging for the topsheet. In another embodiment, the optional cleansing apparatus is integrated into the packaging for the topsheet.

In another embodiment, the present invention provides a method for providing a fresh feeling to a user of an absorbent article, comprising covering a soiled cover of an absorbent article with the topsheet. In yet another embodiment, the present invention provides a method for providing a fresh feeling to a user of an absorbent article, comprising: freshening a target area with a cleansing apparatus and covering a soiled cover of the absorbent article with a topsheet.

The present invention also provides a device for placement between an absorbent product and an undergarment. The device secures and aligns the absorbent product. As a result, the device reduces the movement of the absorbent product, which may prevent leakage.

Thus, in one embodiment of the invention, the device may include a center portion, two end sections, a first protruding end, and a second protruding end. The center portion has an adhesive for attaching the absorbent product to the device. The adhesive is composed of a first adhesive strip and a second adhesive strip. The first adhesive strip is positioned in the center portion to be substantially parallel to a length of the absorbent product and the second adhesive strip is positioned in the center portion to be substantially perpendicular to the length of the absorbent product.

The two end sections attach the device to an undergarment. The two end sections fold around an edge of the undergarment and attach to an outside of the undergarment using an adhesive. The first protruding end extends towards a front of a crotch area of the undergarment and the second protruding end extends towards a back end of the crotch area of the undergarment.

The present invention further provides a target area marker. A target area marker is used to effectively position an absorbent product. Absorbent products include, but are not limited to, feminine hygiene pads, medical dressings, nursing pads, and diapers. The target area marker may be placed on the absorbent product itself; another article, such as an article of clothing, in which the target area marker is attached; and/or a dedicated marking article, such as a sticker. The target area marker may be placed on the absorbent product or other article with a colored “+” mark or any other position guidance shape. The target area marker may be embossed onto the absorbent product or other article. Alternatively, the target area marker may be added to the absorbent product and/or the article of clothing after manufacture using a dedicated marking article. More than one target area marker may be placed on the absorbent product or other article.

These as well as other aspects and advantages will become apparent to those of ordinary skill in the art by reading the following detailed description, with reference where appropriate to the accompanying drawings. Further, it is understood that this summary is merely an example and is not intended to limit the scope of the invention as claimed.

### BRIEF DESCRIPTION OF THE DRAWINGS

The present invention will be understood and appreciated more fully from the following description, taken in conjunction with the drawing in which:

FIG. 1 is a top or bodyside view of an absorbent article for feminine hygiene, a sanitary napkin, according to an exemplary embodiment.

FIG. 2A is a top or bodyside view of an absorbent article for feminine hygiene, a sanitary napkin, according to an exemplary embodiment.

FIG. 2B is a side view of the absorbent article for feminine hygiene, a sanitary napkin, depicted in FIG. 2A, according to an exemplary embodiment.

FIG. 3 is a plan view of a device for securing and aligning absorbent products in accordance with an example;

FIG. 4 is a plan view of the device for securing and aligning absorbent products as depicted in FIG. 3 affixed to an undergarment in accordance with an example.

FIG. 5 is a plan view of an absorbent product affixed to the device for securing and aligning absorbent products as depicted in FIG. 3 in accordance to an example.

FIG. 6 is a side view of the device for securing and aligning absorbent products as depicted in FIG. 3 in accordance with an example.

FIG. 7 is a front view of the undergarment depicted in FIG. 4 with the device depicted in FIG. 3 securing the absorbent product depicted in FIG. 5 in accordance with an example.

FIG. 8 is a packaging diagram for packaging the device for securing and aligning absorbent products depicted in FIG. 3 in accordance with an example.

FIG. 9 is a plan view of a target area marker in one example.

FIG. 10 is a plan view of a target area marker in another example.

FIG. 11 is a plan view of a target area marker in another example.

FIG. 12 is a plan view of a target area marker in another example.

FIG. 13 is a plan view of a target area marker in another example.

FIG. 14 is a plan view of a target area marker in another example.

### DETAILED DESCRIPTION OF THE INVENTION

The invention relates to means for providing a fresh feeling to absorbent article users, a device for securing and aligning absorbent articles, and a target area marker that indicates where to effective place absorbent articles. Each aspect of the invention is described in detail below.

(a) Means To Obtain A Fresh Feeling For Absorbent Article Users

In a typical absorbent article, an absorption layer is enclosed between a bodyside liquid-permeable cover and a liquid-impermeable layer. Optionally, an acquisition layer and a transfer layer may be placed between the cover and the absorption layer.

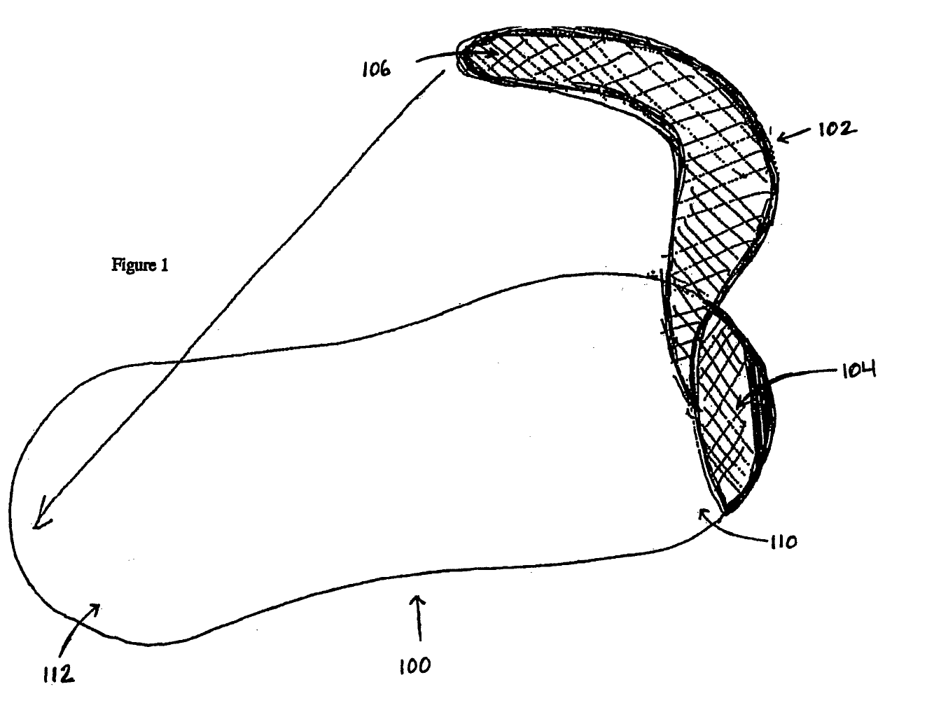

In one embodiment, an additional topsheet for the absorbent article is provided to regenerate a fresh interface between the user and the absorbent article. This additional topsheet can be placed over the existing cover of an absorbent article that is being used by the user, thereby allowing the user to use the absorbent article to its maximum absorbent capacity while still maintaining a fresh interface between the user and the absorbent article. FIG. 1 is a top or bodyside view of an absorbent article for feminine hygiene, a sanitary napkin 100, according to an exemplary embodiment. In FIG. 1, the sanitary napkin 100 is depicted with one end 104 of an additional topsheet 102 partially appended to one end 110 of the sanitary napkin 100. The arrow in FIG. 1 indicates that the unappended second end 106 of the additional topsheet 102 can be aligned with the second end 112 of the sanitary napkin 100 to provide a fresh interface between the user and the sanitary napkin 100.

In this embodiment, the additional topsheet can be integrated into the new absorbent article in an unused form. This unused form can be designed such that the unused topsheet does not or only minimally interferes with the absorptive action of the absorbent article. The unused form can also be designed such that the unused form is virtually undetectable to the user. The integrated unused topsheet can be designed such that the topsheet can be quickly and easily converted into a useable additional topsheet. One way to ensure that an integrated unused topsheet can quickly and easily be converted into a useable form is to provide a means to securely handle the integrated unused topsheet, such as a tab that is accessible in the unused form. By way of example, the unused additional topsheet could be attached to an edge of the absorbent article in a folded form. When this additional topsheet is needed, the user can simply unfold the integrated additional topsheet over the existing cover of the absorbent article to regenerate a fresh interface between the user and the absorbent article. In the case of an absorbent article for feminine hygiene, such as a sanitary napkin, the additional topsheet can be attached in any suitable manner to the front or back edge of the sanitary napkin. In the unused position, this additional topsheet can be folded or otherwise compacted such that it does not or only minimally interferes with the absorptive process, and is virtually undetectable by the user. This unused form of the topsheet could easily be converted by the user to the useable form by simply unfolding or uncompacting the additional topsheet and positioning it over the existing cover of the sanitary napkin, thereby providing a fresh interface between the user and the sanitary napkin.

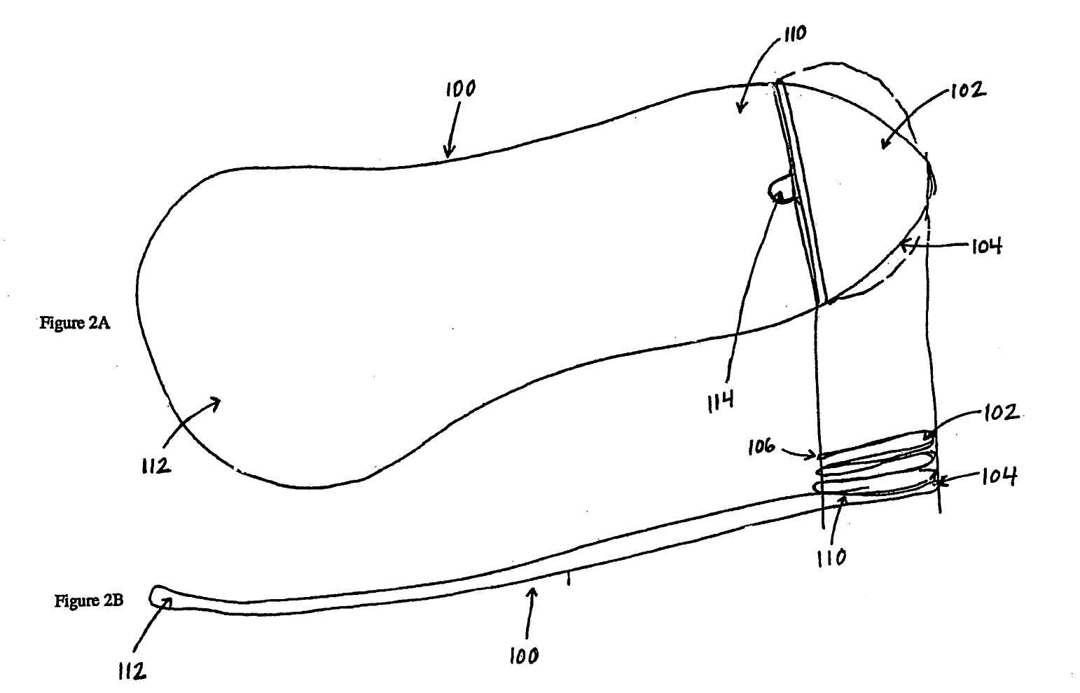

FIG. 2A is a top or bodyside view of an absorbent article for feminine hygiene, a sanitary napkin 100, according to an exemplary embodiment. In FIG. 2A, the sanitary napkin 100 is depicted with an additional topsheet 102 attached at one end 104 of the additional topsheet 102 to one end 110 of the sanitary napkin 100. In this exemplary embodiment, the additional topsheet 102 is folded in the unused position and a tab 114 is integrated in the additional topsheet 102 as a means to quickly and easily convert the additional topsheet 102 from the unused to the useable form. In this embodiment, the user can convert the additional topsheet 102 from the unused to the useable form by extending the additional topsheet 102 over the sanitary napkin 100 such that the second end 106 of the additional topsheet 102 is aligned with the second end 112 of the sanitary napkin 100.

FIG. 2B is a side view of the absorbent article for feminine hygiene, a sanitary napkin 100 depicted in FIG. 2A. In FIG. 2B, the additional topsheet 102 is depicted in an unused form, in this case folded at one end 110 of the sanitary napkin 100. The unused additional topsheet 102 depicted in FIG. 2B can be converted to the useable form by unfolding the additional topsheet 102 and extending it over the existing cover of the sanitary napkin 100 such that the second end 106 of the additional topsheet 102 is aligned with the second end 112 of the sanitary napkin 100.

In this embodiment, the additional topsheet can alternatively be supplied separately from the absorbent articles. In this form, the user can align the separate additional topsheet over the existing cover of the absorbent article to provide a fresh interface between the user and the absorbent article. Alternatively, the user can apply the separate topsheet directly on the user's underpants to improve their fresh feeling while the small amount of discharges, e.g., menses, moves away from the topsheet and absorbed partly by the topsheet and mostly by the underpants itself.

In another embodiment, the additional topsheet can have an adhesive capable of establishing a secure bond with the absorbent article. This adhesive is applied to the side of the additional topsheet intended to contact the absorbent article to secure the additional topsheet to the absorbent article. Moreover, this adhesive can provide a means to maintain an integrated additional topsheet in an unused form. The adhesive can be composed of any material suitable to adhere the additional topsheet to the existing cover of the absorbent article, including but not limited to, water-based pressure-sensitive adhesives such as acrylate adhesives, rapid setting thermoplastic “hot melt” adhesives, or two-sided adhesive tape. The adhesive can be applied to at least a portion of the side of the additional topsheet intended to contact the existing cover of the absorbent article in various patterns including but not limited to parallel longitudinal lines, a line of adhesive following the perimeter of the additional topsheet, or traverse lines of adhesive.

The additional topsheet can be made of any material that allows for rapid acquisition and temporary absorption of the fluids intended to be collected by the absorbent article. In the case of absorbent articles for feminine hygiene, such as sanitary napkins, the materials for the topsheet could include, but are not limited to, cotton fibers, cellulose fibers, synthetic fibers such as polyester or polypropylene, or mixtures of cellulose and synthetic fibers. The topsheet may also be an apertured polymeric film, a perforated plastic film, or any other suitable permeable film material. In practicing this invention, the topsheet is preferably a permeable, hydrophobic film such as aperture polyethylene films such as the ones produced by Tredegar Corp., South Wales, Great Britain; perforated polypropylene nonwovens such as the ones produced by Tredegar Corp.; or nonwoven distribution/acquisition sheets such as the ones produced by Shalag Industries, Upper Galilee, Israel).

The topsheet can contain absorption particles of any suitable material, such as fluff; super absorbent fibers (SAF); treatment material to treat existing conditions or prevent infection (including, but not limited to antibiotics, antifungal compounds, antiviral compounds, and analgesics); diagnostic material (including, but not limited to compounds that test for chemical adulterants, pH indicators, and labeled antibodies); odor control material; liquid absorbing particulate gels; odor absorbing gels; or any other absorbent material. The topsheet can optionally contain any suitable skin freshening material such as aloe vera or gentle moisturing lotions, and any suitable odor control material, such as baking soda, or fragrance.

In another embodiment, an optional cleaning towel or other cleansing apparatus is provided with absorbent articles. This cleansing towel or other cleansing apparatus allows the user of the absorbent article to freshen the target area of the absorbent article prior to the initial use of the absorbent article. This embodiment also allows the user of the absorbent article to freshen the target area prior to interchanging one absorbent article for another one. The cleansing towel or other cleansing apparatus can be attached to the packaging of the absorbent article or integrated into the packaging of the absorbent article. For example, absorbent articles for feminine hygiene, such as sanitary napkins, are typically packaged separately. In this embodiment, a separately packaged cleansing towel or other cleansing apparatus could be attached to the outside or inside of the individual sanitary napkin package. Alternatively, the cleansing towel or other cleansing apparatus could be integrated into the individual sanitary napkin package. This would provide the user of the sanitary napkin with a means of freshening the perineal area prior to the initial use of the sanitary napkin or when interchanging one sanitary napkin for another one.

The topsheet of the invention can be produced in any color or combination of colors. Furthermore, the topsheet can be produced in any suitable shape and size.

(b) Device For Securing And Aligning Absorbent Products

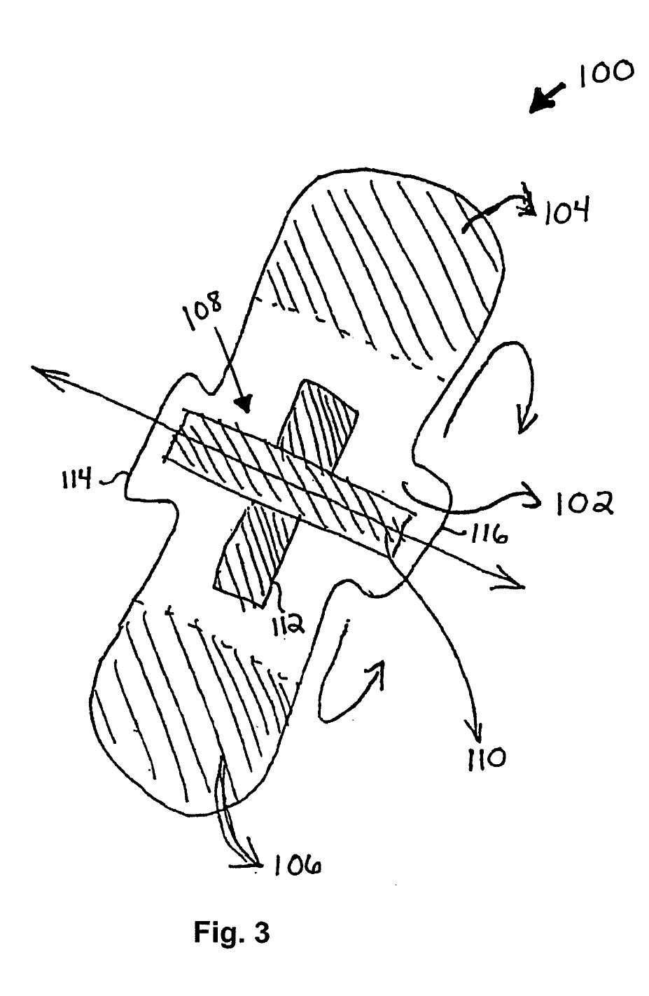

FIG. 3 is a plan view of a device 100 for securing and aligning absorbent products. The device 100 includes a center portion 102 and two end sections 104, 106. The device 100 may be composed of a variety of liquid impermeable materials. Additionally or alternatively, the device 100 may be covered with smooth hydrophobic materials. The size and shape of the device 100 may vary according to planned usage.

Adhesive 108 may be added to the center portion 102. The adhesive 108 may be composed of a variety of materials including, but not limited to, water-based pressure-sensitive adhesives, such as acrylate adhesives; rapid setting thermoplastic hot-melt adhesives; body-sensitive adhesives, or double-sided tape. Alternatively, a Velcro® material may be added to the center portion 102. The Velcro® material may be used on the device 100 with an absorbent product also having a Velcro® layer.

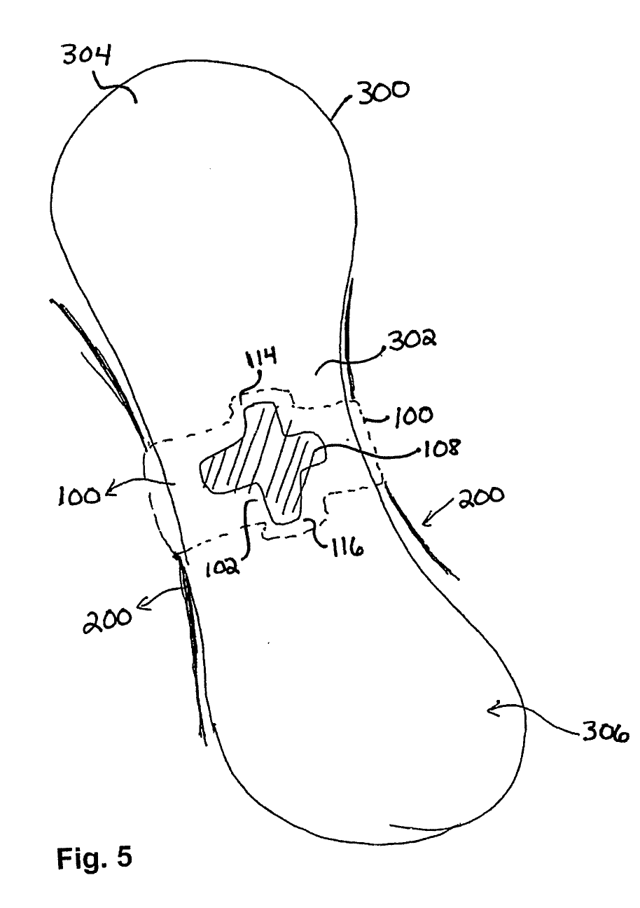

The center portion 102 may support an absorbent product as further described with reference to FIG. 5. The center portion 102 may include the adhesive 108 for attaching the absorbent product. As depicted in FIG. 3, the adhesive 108 may be composed of two adhesive strips 110, 112. The adhesive strip 110 may be positioned on the center portion 102 approximately in the same direction (i.e., parallel to) as the length of the absorbent product to be attached to the device 100. The adhesive strip 112 may be positioned on the center portion 102 substantially perpendicular to the length of the absorbent product to be attached to the device. More than or less than two adhesive strips may be used as well.

The center portion 102 may include two protruding ends 114, 116. The first protruding end 114 may extend towards a front of a crotch area of an undergarment, while the second protruding end 116 may extend towards a back of the crotch area of the undergarment. The shape and/or the length of the protruding ends 114, 116 may depend on the type and/or size of the undergarment that the device 100 will be attached to.

The two end sections 104, 106 may be used for attaching the device 100 to an undergarment. The two end sections 104, 106 may be described as “flaps” or “wings.” The two end sections 104, 106 may fold around an edge of the undergarment and attach to the outside of the undergarment. The two end sections 104, 106 may be attached using an adhesive. The adhesive may be the same or a different type of adhesive in the center portion 102 of the device 100.

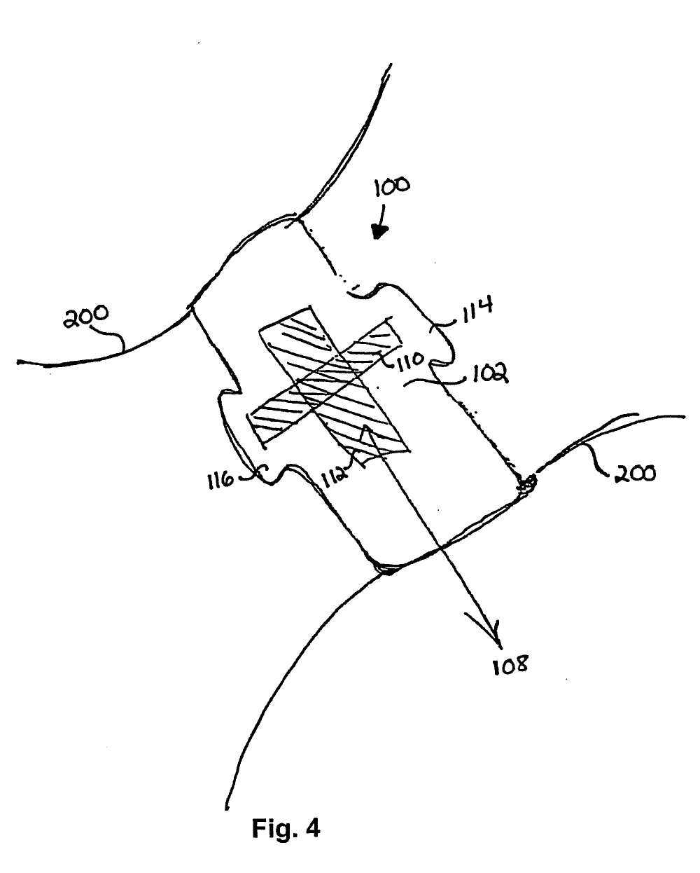

FIG. 4 is a plan view of the device 100 for securing absorbent products affixed to an undergarment 200. As seen in FIG. 4, the center portion 102 for supporting an absorbent product is positioned in the inside crotch area of the undergarment 200. The two end sections 104, 106 are not visible in FIG. 4 as the two end sections 104, 106 have been folded around the edge of the undergarment 200 and attached to the outside crotch area of the undergarment 200. The two end sections 104, 106 secure the device 100 to the undergarment 200.

FIG. 5 is a plan view of an absorbent product 300 affixed to the device 100 for securing absorbent products. The absorbent product 300 may have a center portion 302 and two end sections 304, 306. The center portion 302 of the absorbent product 300 may be attached with the adhesive 108 to the center portion 102 of the device 100. The first end section 304 may extend towards the front of the crotch area of the undergarment 200, while the second end section 306 may extend towards the back of the crotch area of the undergarment 200.

The device 100 is shown in FIG. 5 with dashed lines to indicate that the absorbent product 300 is placed on the device 100. The method of placing the absorbent product 300 on the device 100 may vary. Preferably, a user places the device 100 in the crotch area of the undergarment 200 first, then places the absorbent product 300 near the most appropriate location of the user's body, then brings the undergarment 200 towards the absorbent product 300 attaching the device 100 to the absorbent product 300. However, other methods of attaching the device 100, the undergarment 200, and the absorbent product 300 may be used.

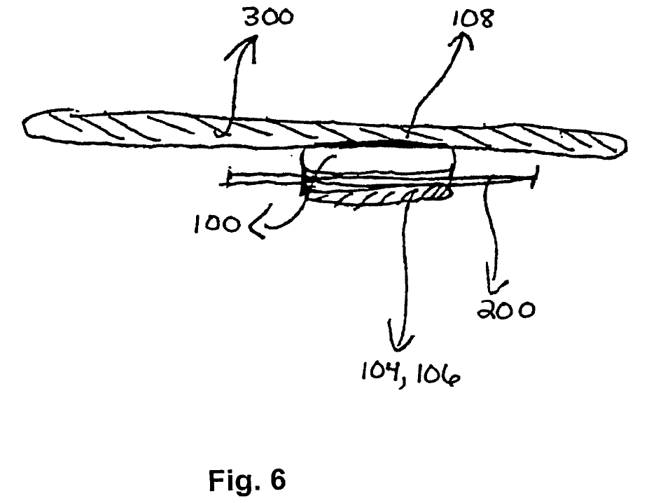

FIG. 6 is a side view of the device 100 for securing absorbent products. The side view depicts that the absorbent product 300 is attached to the top of the device 100 with the adhesive 108. Further, the device 100 is attached to the inside of the undergarment 200 with the two end sections 104, 106.

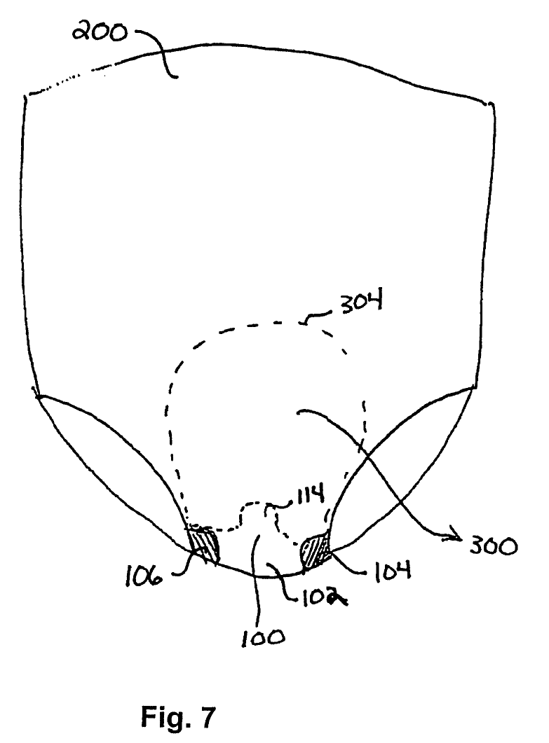

FIG. 7 is a front view of the undergarment 200 with the device 100 securing the absorbent product 300. The absorbent product 300 and part of the device 100 are shown in FIG. 7 with dashed lines to indicate that the absorbent product 300 and part of the device 100 are inside the undergarment 200 and are not visible in this view. Further, the first end 304 of the absorbent product 300 and the first protruding end 114 of the center portion 102 of the device 100 are depicted in FIG. 7, as the first end 304 and the first protruding end 114 extend towards the front of the crotch area of the undergarment 200. The second end 306 of the absorbent product 300 and the second protruding end 116 of the center portion 102 of the device 100 are not depicted in FIG. 7 as the second end 306 and the second protruding end 116 extend towards the back of the crotch area of the undergarment 200. The two end sections 104, 106 are located on the outside 200 and are visible in this view.

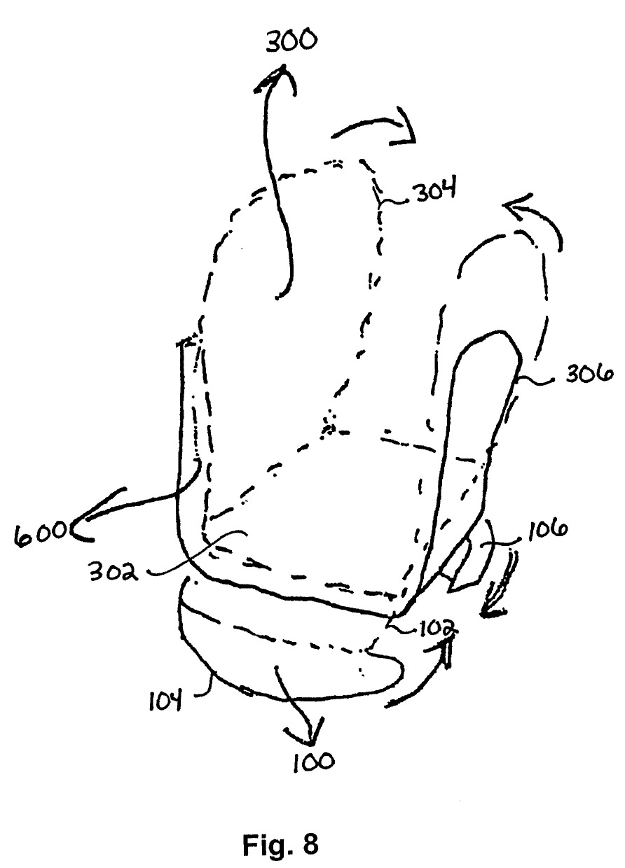

FIG. 8 is a packaging diagram for packaging the device 100 for securing absorbent products. The device 100 and the absorbent product 300 may be supplied together or separately. The device 100 may be supplied separately from the absorbent product 300 as the device 100 may be reusable for more than one absorbent product. For example, one device 100 may be used to secure four different feminine hygiene pads in a single day. When the device 100 is supplied separately, the device 100 may be folded such that the two end sections 104, 106 of the device 100 are folded under or above the center portion 102 of the device 100.

When the device 100 and the absorbent product 300 are supplied together the device 100 and the absorbent product 300 may be packaged as depicted in FIG. 8. The device 100 may be folded such that the two end sections 104, 106 of the device 100 are folded under the center portion 102 of the device 100. The absorbent product 300 may be folded such that the two end sections 304, 306 of the absorbent product 300 may be folded above the center portion 302 of the absorbent product 300. The device 100 and the absorbent product 300 may be packaged so that the side of the device 100 that is attached to the undergarment 200 and the side of the absorbent product 300 that is attached to the device 100 are adjacent to each other. Further, the folds of the end sections 104, 106 of the device 100 and the folds of the end sections 304, 306 of the absorbent product 300 may be on opposite ends. A common release strip 600 may be used to cover the adhesive on the bottom of the absorbent product 300 and the adhesive strip 110 on the center portion 102 of the device 100.

The device 100 secures and aligns the absorbent product 300. For example, the device 100 may be used with absorbent products having a width greater than the crotch area of the undergarment 200. In this way, a large absorbent product 300, such as an incontinence pad, can be secured and aligned. As a result, the device 100 reduces the movement of the absorbent product 300, which may prevent leakage.

(c) Target Area Marker For Absorbent Products

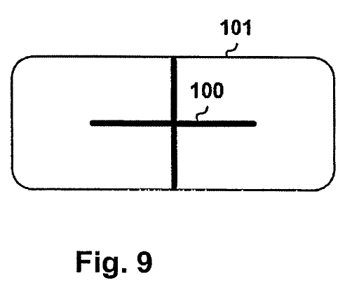

FIG. 9 is a plan view of a target area marker 100. The target area marker 100 is depicted in FIG. 9 on a feminine hygiene pad 101; however, as will be fully discussed below, this is only one example of where the target area marker 100 may be located.

The target area marker 100 may be placed on a variety of articles. As shown in FIG. 9, the target area marker 100 may be place on the absorbent product itself. For example, the target area marker 100 may be placed on the feminine hygiene pad 101, a protective undergarment, a medical dressing, a nursing pad, a diaper, underwear, and so on.

In another embodiment, the target area marker 100 may be placed on an article, such as an article of clothing, in which an absorbent product is to be attached. The article of clothing may be for a man or a woman. Additionally, the article of clothing may be for an individual of any age, such as an adult, a child, or an infant. As an example, the target area marker 100 may be placed in the crotch area on the inside of a pair of pants.

In another embodiment, the target area marker 100 may be placed on a dedicated marking article, such as a sticker. In this embodiment, the target area marker 100 may be easily added to an absorbent product and/or an article of clothing after manufacture. In this manner, the absorbent product and/or article of clothing may be modified to indicate where to effectively place absorbent products.

The target area marker 100 may be placed on an article using a colored “+” or cross mark. Any variety of colors may be used so as to distinguish the target area marker 100 from the article. For example, if the article is white, a black target area marker 100 may be placed on the article. Additionally, a variety of shapes may be used to guide the placement of the mark. For example, a circle may be used on a nursing pad to correspond to the location of a woman's nipple. As a result, the absorbent product may be effectively placed by sight. The colors can be made of any suitable material such as pigments, dyes, or colored polymers, preferably of the type that will not stain or irritate skin.

In another embodiment, the target area marker 100 may be embossed onto an article. As a result the target area marker 100 may have a distinct feel. The embossed target area marker 100 may provide placement guidance to a person who has difficulty seeing or is trying to position an absorbent product in an area that is not well lit. As a result, the absorbent product may be effectively placed by touch.

As shown in FIG. 9, the “+” mark extends from one edge of the feminine hygiene pad 101 to the other. However, the size of the target area marker 100 may be adjusted to provide adequate guidance for effective placement of an absorbent product.

The articles may be disposable or washable. Accordingly, the target area marker 100 may be affixed to the article in an appropriate manner. For example, the target area marker 100 may be affixed on the article in a manner that will withstand repeated washings when placed on washable articles. If the article is disposable, the target area marker 100 may not be affixed to the article in a manner to withstand repeated washings.

The target area marker 100 may be placed on the side of the absorbent product facing an individual's body when the absorbent product is in place. The target area marker 100 may also be placed on the side of the absorbent product facing the individual's clothing when the absorbent product is in place. When the target area marker 100 is placed on the side of the absorbent product facing the individual's body, the individual may place the target area marker 100 so as to touch or be adjacent to the appropriate place on the individual's body. The individual may then secure the absorbent product with their clothing or other means. For example, a woman may place a target area marker 100 located on the feminine hygiene pad 101 closest to her source of menses. The woman may then secure the feminine hygiene pad 101 with her underpants.

When the target area marker 100 is placed on the side of the absorbent product facing the individual's clothing, the individual may place the target area marker 100 touching the appropriate place on the individual's clothing. In this manner, the absorbent product may be effectively placed when the clothing is not on the individual. Effectively placing the absorbent product on the clothing prior to donning the clothing may be useful when the individual placing the absorbent product on the clothing is not the same individual that will be wearing the clothing. Further, effectively placing the absorbent product on the clothing prior to donning the clothing may be useful when the individual placing the absorbent product on the clothing has difficulty dressing themselves.

The target area marker 100 may also be placed on an article of clothing. The article of clothing may be purchased with the target area marker 100 already affixed to the garment. For example, nursing bras may be purchased with the target area marker 100 already in place. A woman may be able to effectively place a nursing pad on the nursing bra using the target area marker 100.

Alternatively, the target area marker 100 may be added to the article of clothing after the article is purchased. As an example, the target area marker 100 may be placed in the undergarments of a nursing home patient after the patient is admitted to the facility. The staff at the nursing home may determine the most effective placement of incontinence pads that are used at their facility. As a result, the nursing home can maximize the usage of the incontinence pads, which may reduce waste and result in a cost savings. Additionally, leakage problems may be reduced, which may also lead to costs savings due to reduced staff time needed to clean after a leak has occurred.

Additionally, the target area marker 100 may be placed on both the absorbent product and the article of clothing. In this embodiment, the individual may align both target area markers 100 with each other to effectively positioning the absorbent product on the article of clothing.

More than one target area marker 100 may be placed on one article. This may be useful when an article has more than one purpose. For example, a hygiene pad may be used for both menses and incontinence. There may be two target area markers 100 on the hygiene pad, one for menses and one for incontinence. The two target area markers 100 may have different colors and/or shapes to distinguish the two uses of the hygiene pad. The hygiene pad may be effectively positioned based on its current use.

Additionally, more than one target area marker 100 on an article may be useful for certain types of clothing. For example, pants may be marked with a target area marker 100 in several locations. The target area markers 100 may be placed so that an absorbent product may be effectively positioned in the pants, such as on both sides of the leg openings.

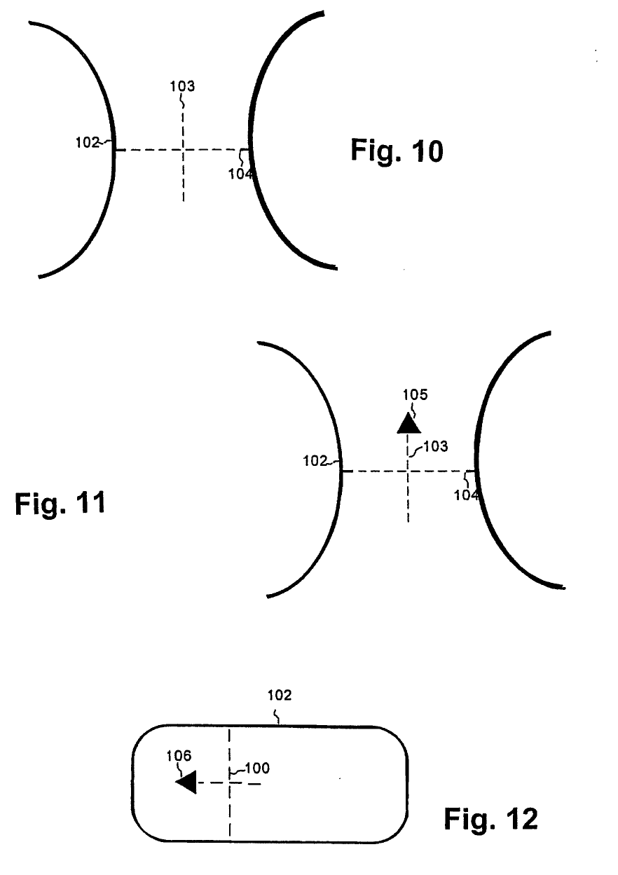

FIG. 10 is a plan view of the target area marker 103. In this example, the target area marker 103 is located in a crotch area of a female's undergarment 102. The target area marker 103 may include two lines 104 forming a cross. One of the lines 104 may be thicker at each end closest to an internal side of leg openings of the undergarment102. The thicker ends of the line 104 may provide guidance for aligning an absorbent product on the undergarment 102 from side to side. The thinner segments of the lines 104 may provide guidance for aligning the absorbent product on the undergarment 102 from front to back. The target area marker 103 may be added to the undergarment 102 at the time of manufacture. Alternatively, the target area marker 103 may be added to the undergarment 102 after manufacture using a dedicated marking article.

FIG. 11 is a plan view of the target area marker 103 having an arrow head 105 at an end of one of the lines 104. The arrow head 105 is located on the line 104 providing guidance for aligning the absorbent product on the undergarment 102 from front to back. The arrow head 105 may provide directional guidance indicating where the front of the absorbent product should be placed on the undergarment 102. Using an arrowhead 105 on the target area marker 103 may be useful when a person has difficulty determining which end of the absorbent product should be placed at the front or back sides of their bodies.

FIG. 12 is a plan view of the target area marker 100 depicted in FIG. 9 having an arrow head 106 at the end of one of the lines of the target area marker 100. The arrow head 106 has the same function as the arrow head 105 as described with reference with FIG. 11. FIG. 12 shows that the arrow head 106 can be used with a target area marker 100 located on a feminine hygiene pad 101, as well as an undergarment.

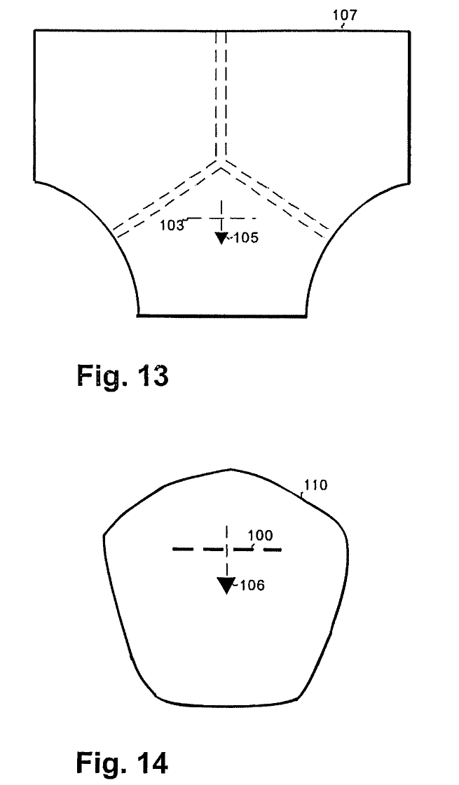

FIG. 13 is a plan view of the target area marker 103 located on a male undergarment 107. The target area marker 103 includes the arrow head 105. FIG. 13 shows that the target area marker 103 may be placed on male undergarments, as well as female undergarments.

FIG. 14 is a plan view of the target area marker 100 located on a male absorbent product 110. The target area marker 100 includes the arrow head 106. FIG. 14 shows that the target area marker 100 may be placed on absorbent products designed for males, as well as females.

By effectively placing the absorbent product, the absorption capacity of the product may be maximized. As a result, waste, costs, and leakage problems may be reduced.

It will be appreciated by persons skilled in the art that the present invention is not limited by what has been particularly shown and described herein. Rather the scope of the invention includes variations and modification of the various features described in the specification and shown in the drawings which may occur to a person of ordinary skill in the art upon reading the foregoing description.

1. A stand alone attachable, liquid permeable topsheet article to cover absorbent articles, the topsheet containing an adhesive to attach the topsheet to the absorbent article.

2. The stand alone topsheet of claim 1, wherein the absorbent article is an undergarment, an absorbent pad, a protective undergarment, a diaper, a nursing pad, or a medical dressing.

3. A stand alone attachable topsheet article comprising:
a topsheet having opposed inwardly and outwardly oriented faces;
a liquid permeable cover positioned on the inwardly oriented face of the topsheet;
a layer of absorption particles positioned on the outwardly oriented face of the topsheet; and
an adhesive layer on the outwardly oriented face of the topsheet.

4. The stand alone topsheet article of claim 3, further comprising at least one of a freshening material, an odor-control material, or both.

5. The stand alone topsheet article of claims 1 or 3, wherein topsheet can be produced in white or non-white color.

6. The stand alone topsheet article of claims 1 or 3,wherein the topsheet article is enclosed in a packaging.

7. The stand alone topsheet article of claim 6, wherein a cleansing apparatus is additionally provided with the topsheet article.

8. The stand alone topsheet article of claim 7, wherein the cleansing apparatus is attached to the packaging for the topsheet article.

9. The stand alone topsheet article of claim 7, wherein the cleansing apparatus is integrated into the packaging for the topsheet article.

10. An absorbent article comprising:
a liquid permeable cover;
a liquid impermeable backsheet joined to the cover, said backsheet having opposed inwardly and outwardly oriented faces;
an adhesive layer on the outwardly oriented face of the backsheet;
an absorption layer positioned between the cover and the backsheet; and
a liquid permeable topsheet capable of covering at least part of the cover.

11. The absorbent article of claim 10, wherein the topsheet is integrated into the absorbent article by fixedly attaching a portion of the topsheet to a portion of the cover.

12. The absorbent article of claim 10, wherein the topsheet is provided in a condensed state in a new absorbent article, such that the topsheet does not fully cover the cover of the absorbent article.

13. The absorbent article of claim 12, wherein the topsheet further comprises a tab for engagement and alignment of the folded topsheet over the cover.

14. The absorbent article of claim 10, wherein the topsheet contains an adhesive for securing the topsheet to the cover of the absorbent article.

15. The absorbent article of claim 10,wherein the absorbent article is enclosed in a packaging.

16. The absorbent article of claim 15, wherein a cleansing apparatus is additionally provided with the absorbent article.

17. The absorbent article of claim 16, wherein the cleansing apparatus is attached to the packaging for the absorbent article.

18. The absorbent article of claim 16, wherein the cleansing apparatus is integrated into the packaging for the absorbent article.

19. A method for providing a fresh feeling to a user of an absorbent article, comprising covering a soiled cover of an absorbent article of claim 10 with the topsheet.

20. A method for providing a fresh feeling to a user of an absorbent article, comprising:
a) freshening a target area with the cleansing apparatus of any of claims 7-9 and 16-18; and
b) covering a soiled cover of the absorbent article with the topsheet of any one of claims 1, 3, or 10.

21. A device for securing and aligning an absorbent product, comprising in combination:
a center portion having adhesive for attaching the absorbent product to the device; and
two end sections for attaching the device to an undergarment, wherein the absorbent product is secured to and aligned with the undergarment when using the device.

22. The device of claim 21, wherein the device is composed of liquid impermeable materials.

23. The device of claim 21, wherein the adhesive is a water-based pressure-sensitive adhesive selected from the group consisting of an acrylate adhesive, a rapid setting thermoplastic hot-melt adhesive, a body-sensitive adhesive, and a double-sided tape.

24. The device of claim 21, wherein the adhesive includes a Velcro® material.

25. The device of claim 21, wherein the adhesive is composed of a first adhesive strip and a second adhesive strip.

26. The device of claim 25, wherein the first adhesive strip is positioned in the center portion to be substantially parallel to a length of the absorbent product and the second adhesive strip is positioned in the center portion to be substantially perpendicular to the length of the absorbent product.

27. The device of claim 21, wherein the center portion includes a first protruding end extending towards a front of a crotch area of the undergarment when attached to the undergarment and a second protruding end extending towards a back end of the crotch area of the undergarment when attached to the undergarment.

28. The device of claim 21, wherein the two end sections fold around an edge of the undergarment and attach outside of the undergarment.

29. The device of claim 28, wherein the two end sections are attached to the outside of the undergarment using an adhesive.

30. The device of claim 21, wherein the device is supplied with the absorbent product.

31. A device for securing and aligning an absorbent product, comprising in combination:
a center portion having adhesive for attaching the absorbent product to the device, wherein the adhesive is composed of a first adhesive strip and a second adhesive strip, and wherein the first adhesive strip is positioned in the center portion to be substantially parallel to a length of the absorbent product and the second adhesive strip is positioned in the center portion to be substantially perpendicular to the length of the absorbent product; and
two end sections for attaching the device to an undergarment, wherein the two end sections fold around an edge of the undergarment and attach outside of the undergarment using an adhesive, and wherein the absorbent product is secured to and aligned with the undergarment when using the device.

32. A method for securing and aligning an absorbent product to an undergarment, comprising in combination:
placing a device on the undergarment, wherein the device includes two end sections for attaching to the undergarment and a center portion having an adhesive for attaching the absorbent product;
attaching the device to the undergarment using the two end sections; and
placing the absorbent product on the adhesive located on the center portion of the device, wherein the absorbent product is secured to and aligned with the device.

33. The method of claim 32, wherein the device is composed of liquid impermeable materials.

34. The method of claim 32, wherein the adhesive is a water-based pressure-sensitive adhesive selected from the group consisting of an acrylate adhesive, a rapid setting thermoplastic hot-melt adhesive, a body-sensitive adhesive, and a double-sided tape.

35. The method of claim 32, wherein the adhesive includes a Velcro material.

36. The method of claim 32, wherein the adhesive is composed of a first adhesive strip and a second adhesive strip.

37. The method of claim 36, wherein the first adhesive strip is positioned in the center portion to be substantially parallel to a length of the absorbent product and the second adhesive strip is positioned in the center portion to be substantially perpendicular to the length of the absorbent product.

38. The method of claim 32, wherein the center portion includes a first protruding end extending towards a front of a crotch area of the undergarment when attached to the undergarment and a second protruding end extending towards a back end of the crotch area of the undergarment when attached to the undergarment.

39. The method of claim 32, wherein attaching the device to the undergarment includes folding the two end sections around an edge of the undergarment and attaching the two end sections outside of the undergarment.

40. The method of claim 39, wherein attaching the two sections outside of the undergarment includes attaching the two end sections using an adhesive.

41. A target area marker comprising at least one mark that is placed on an article, wherein the mark indicates where to effectively place an absorbent product.

42. The target area marker of claim 41, wherein the mark has a color that is distinguishable from the article.

43. The target area marker of claim 41, wherein the mark has a shape that guides placement of the absorbent product on the article.

44. The target area marker of claim 41, wherein the mark includes an arrow that provides directional guidance for placing the absorbent product.

45. The target area marker of claim 41, wherein the mark is embossed onto the article.

46. The target area marker of claim 41, wherein the article is selected from the group consisting of the absorbent product, an article of clothing in which the absorbent product is attached, and a dedicated marking article.

47. The target area marker of claim 41, wherein the absorbent product is selected from the group consisting of a feminine hygiene pad, a protective undergarment, a medical dressing, a nursing pad, and a diaper.

48. The target area marker of claim 41, wherein the mark is placed on a side of the article that faces an individual when the absorbent product is in place.

49. The target area marker of claim 41, wherein the mark is placed on a side of the article that faces an individual's clothing when the absorbent product is in place.

50. The target area marker of claim 41, wherein the mark is placed on the article during manufacture.

51. The target area marker of claim 41, wherein the mark is placed on the article after manufacture.

52. A target area marker comprising at least one mark that is placed on an article, wherein the mark has a color that is distinguishable from the article and a shape that guides placement of the mark on the article, wherein the article is selected from the group consisting of an absorbent product, an article of clothing in which the absorbent product is attached, and a dedicated marking article, and wherein the mark indicates where to effectively place the absorbent product.

53. A method for effectively placing absorbent products, comprising of placing at least one mark on an article, wherein the mark indicates where to effectively place an absorbent product.

54. The method of claim 53, wherein the mark has a color that is distinguishable from the article.

55. The method of claim 53, wherein the mark has a shape that guides placement of the absorbent product on the article.

56. The method of claim 53, wherein the mark includes an arrow that provides directional guidance for placing the absorbent product.

57. The method of claim 53, wherein placing the mark includes embossed the mark onto the article.

58. The method of claim 53, wherein the article is selected from the group consisting of the absorbent product, an article of clothing in which the absorbent product is attached, and a dedicated marking article.

59. The method of claim 53, wherein the absorbent product is selected from the group consisting of a feminine hygiene pad, a protective undergarment, a medical dressing, a nursing pad, and a diaper.

60. The method of claim 53, wherein placing the mark includes placing the mark on a side of the article that faces an individual when the absorbent product is in place.

61. The method of claim 53, wherein placing the mark includes placing the mark on a side of the article that faces an individual's clothing when the absorbent product is in place.

62. The method of claim 53, wherein placing the mark on the article occurs during manufacture.

63. The method of claim 53, wherein placing the mark on the article occurs after manufacture.

64. An article comprising a target area marker, wherein the target area marker indicates where to effectively place an absorbent product.

65. The article of claim 64, wherein the target area marker has a color that is distinguishable from the article.

66. The article of claim 64, wherein the target area marker has a shape that guides placement of the absorbent product on the article.

67. The article of claim 64, wherein the target area marker includes an arrow that provides directional guidance for placing the absorbent product.

68. The article of claim 64, wherein the target area marker is embossed onto the article.

69. The article of claim 64, wherein the article is selected from the group consisting of the absorbent product, an article of clothing in which the absorbent product is attached, and a dedicated marking article.

70. The article of claim 64, wherein the absorbent product is selected from the group consisting of a feminine hygiene pad, a protective undergarment, a medical dressing, a nursing pad, and a diaper.

71. The article of claim 64, wherein the target area marker is placed on a side of the article that faces an individual when the absorbent product is in place.

72. The article of claim 64, wherein the target area marker is placed on a side of the article that faces an individual's clothing when the absorbent product is in place.
